(19) United States
(12) Patent Application Publication
(10) Pub. No.: US 2016/0282353 A1
(43) Pub. Date: Sep. 29, 2016

(54) SYSTEMS AND METHODS OF DETECTING MALIGNANT CELLS

(71) Applicant: IVDiagnostics, Inc., Crown Point, IN (US)

(72) Inventors: Christopher M. Clemens, Crown Point, IN (US); Joseph R. Firca, Lake Bluff, IL (US)

(21) Appl. No.: 15/082,532

(22) Filed: Mar. 28, 2016

Related U.S. Application Data
(60) Provisional application No. 62/138,558, filed on Mar. 26, 2015.

(51) Int. Cl. G01N 33/574; G01N 15/06

(52) U.S. Cl. CPC G01N 33/57492; G01N 15/0656; G01N 2333/70589; G01N 2015/0693; G01N 2015/0069

(57) ABSTRACT

Provided is an assay system and method that includes binding a conjugate to a target receptor on a surface of malignant blood cells included in the liquid medium to form labeled malignant blood cells. The liquid medium including the labeled malignant blood cells is exposed to a magnetic field to separate the labeled malignant blood cells from unlabeled blood cells in the liquid medium. In the presence of the magnet field, at least a portion of the liquid medium is removed to isolate the labeled malignant blood cells separated by the magnetic field. A sample comprising at least a portion of the labeled malignant blood cells separated by the magnetic field is then introduced into a flow cytometer to quantify the labeled malignant blood cells present in the sample.

### CROSS-REFERENCE TO RELATED APPLICATIONS

This application claims the benefit of U.S. Provisional Application No. 62/138,558, filed Mar. 26, 2015, which is incorporated in its entirety herein by reference.

### BACKGROUND OF THE INVENTION

1. Field of the Invention

The present subject matter relates generally to systems and methods for detecting malignant cells by binding fluorescent magnetic microspheres and non-fluorescent magnetic microspheres to abnormal biomarkers.

2. Description of Related Art

Biomedical research has evolved to include identifying disease-related genes using assay chemistry. For example, microarrays include a plurality of target-specific receptors to detect a specific DNA sequence. In another example, suspension array technology (SAT) provides a high-throughput assay chemistry by utilizing encoded microparticles in combination with flow-based analysis cytometry.

SAT allows for the simultaneous testing of multiple gene variants through the use of microsphere beads as each type of microsphere bead has a unique identification based on variations in optical properties, such as a fluorescent source. Similar to the flat microarrays, an appropriate receptor molecule, such as DNA oligonucleotide probes, antibodies, or other proteins, is attached to differently labeled microspheres. The microspheres bound with the receptor molecules are typically detected by optical labeled targets to determine the relative abundance of each target in the sample.

It would be advantageous to provide multimodal systems and methods for detecting and isolating entire target cells.

### BRIEF SUMMARY OF THE INVENTION

It has been determined that the quantification of target samples hybridized to an optical identifier can be achieved by comparing the relative intensity of target markers in one specimen containing the optical identifier to the intensity of target markers in another specimen of the optical identifier using flow cytometry. Flow cytometry is a biomarker detection system that applies passing suspended cells in a stream of fluid through an electric detection device. Flow cytometers are able to analyze several thousand particles every second and can separate and isolate particles having specified properties.

Magnetic particles can be coated with biologically-active materials that will cause them to bond strongly with specific targets, including proteins, viruses, and DNA fragments. These magnetic particles become objects used to immobilize the bio-target, after which the magnetic particles may be isolated using a magnetic field.

According to one aspect, the present disclosure provides systems and methods for detecting malignant cells by binding fluorescent magnetic microspheres to abnormal biomarkers associated with malignant cells. Various examples of the systems and methods are provided herein.

In neoplastic disease, malignant cells often express biomarkers, such as antigens, receptors, or other cell surface structures, at levels not found on normal (non-malignant) cells. Detection of these abnormal biomarker expression profiles provide a method by which malignant cells can be identified, quantitated and monitored.

For example, the CD45 antigen (leukocyte common antigen) is a receptor-linked protein tyrosine phosphatase biomarker that is expressed on all leukocytes. For subjects afflicted with a neoplastic disease however, CD45 antigen expression is expressed at significantly greater levels than for healthy subjects not afflicted with the neoplastic disease. Therefore, detection of abnormal CD45 expression profiles provides a valuable method by which the presence of malignant cells may be detected.

According to another aspect, the present systems and methods include binding of fluorescent magnetic microspheres (e.g., 0.3 μm microparticles) derivatized with anti CD45 scFv (0.3 μm-Fl-anti-CD45) to viable human leukocytes in whole blood followed by isolation and recovery of the labeled leukocytes by magnetic separation. An scFv is a single-chain variable fragment (scFv) that is not actually a fragment of an antibody, but instead is a fusion protein of the variable regions of the heavy (VH) and light chains (VL) of immunoglobulins. This protein retains the specificity of the original immunoglobulin, despite removal of the constant regions and the introduction of a linker. For example, a leukocyte fraction obtained from whole human blood may be incubated with 0.3 μm-Fl-anti-CD45 magnetic microspheres to bind the microspheres to CD45, forming labeled leukocytes. The resulting 0.3 μm-Fl-anti-CD45-labeled leukocytes are then isolated by the process of magnetic separation.

According to another aspect, the present method may include (a) labeling of leukocytes by incubation with 0.3 μm fluorescent magnetic microspheres derivatized with anti CD45 scFv, (b) isolating of the labeled leukocytes by magnetic separation, and (c) measuring of the labeled leukocytes by flow cytometry. In addition to, or in lieu of scFv proteins, the fluorescent microspheres may be derivatized with nucleic acid polymers such as DNA and RNA, peptides, aptamers and other molecules demonstrating specificity to target molecules such as CD45, Nodal, DCKL1, folic acid receptors (FR) and other biomarkers on the CTCs, for example.

According to another aspect, the present disclosure provides an assay method that includes contacting human blood cells in a liquid medium with a conjugate comprising a fluorescent magnetic particle derivatized with a molecule that demonstrates specificity for a target on a surface of malignant blood cells included in the liquid medium to form labeled malignant blood cells. The liquid medium including the labeled malignant blood cells is exposed to a magnetic field to separate the labeled malignant blood cells from unlabeled blood cells in the liquid medium. In the presence of the magnet field, at least a portion of the liquid medium is removed to isolate the labeled malignant blood cells separated by the magnetic field. A sample comprising at least a portion of the labeled malignant blood cells separated by the magnetic field is then introduced into a flow cytometer to quantify the labeled malignant blood cells present in the sample.

An advantage of the present system and method includes detection and isolation of entire malignant cells. In other words, the present systems and methods are advancements over merely detecting enzymes, DNA fragments, antibodies, antigens and other small biomolecules.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

The above summary presents a simplified summary in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

### BRIEF DESCRIPTION OF SEVERAL VIEWS OF THE DRAWING

The invention may take physical form in certain parts and arrangement of parts, embodiments of which will be described in detail in this specification and illustrated in the accompanying drawings which form a part hereof and wherein:

FIG. 1 shows an embodiment of a magnetic separation device with a received sample tube containing a liquid suspension of leukocytes bonded with fluorescent magnetic microspheres;

FIG. 2A is a cross sectional view of the magnetic separation device taken along line 2-2 in FIG. 1;

FIG. 2B shows an alternate embodiment of a magnet for magnetic separation of magnetic microparticles bound to target cells from a suspension liquid;

FIG. 3A is a flow diagram graphically depicting an embodiment of a general assay method;

FIG. 3B is a flow diagram graphically depicting an embodiment of a magnetic separation method;

FIG. 4 shows the results of the flow cytometry analysis of 0.3 μm-anti-CD45 in 1% milk, following a 5 minute magnetic separation process;

FIG. 5 shows the results of the flow cytometry analysis of 0.3 μm-anti-CD45 in 1% milk, following a 10 minute magnetic separation process

FIGS. 6-11 show the results of the flow cytometry analysis of Samples for Example 5;

FIGS. 12-19 show the results of the flow cytometry analysis of Samples for Example 6;

FIGS. 20-23 show the results of the flow cytometry analysis of Samples for Example 7;

FIGS. 24-28 show the results of the flow cytometry analysis of Samples for Example 8;

FIGS. 29-35 show the results of the flow cytometry analysis of Samples for Example 9; and

FIGS. 36 and 37 show the results of the flow cytometry analysis of Samples for Example 10.

### DETAILED DESCRIPTION OF THE INVENTION

Certain terminology is used herein for convenience only and is not to be taken as a limitation on the present invention. Relative language used herein is best understood with reference to the drawings, in which like numerals are used to identify like or similar items. Further, in the drawings, certain features may be shown in somewhat schematic form.

The present systems and methods may involve (a) labeling of leukocytes by incubation with fluorescent magnetic microspheres conjugated to anti-human CD45 scFv or other suitable antibody with an affinity for a biomarker desired to be detected. As stated previously, other molecules may be substituted for scFvs; (b) isolating the labeled leukocytes by magnetic separation; and (c) measuring of the labeled leukocytes by flow cytometry or other suitable batch or flow analytic device. For example, fluorescent magnetic microspheres (e.g., 0.3 μm microparticles, but any sizes of microparticles can be used, optionally within a range of sizes from about 0.2 μm to about 0.6 μm, etc.) conjugated with anti CD45 scFv may bind to viable human leukocytes in whole blood, forming labeled leukocytes. The labeled leukocytes may be isolated by magnetic separation.

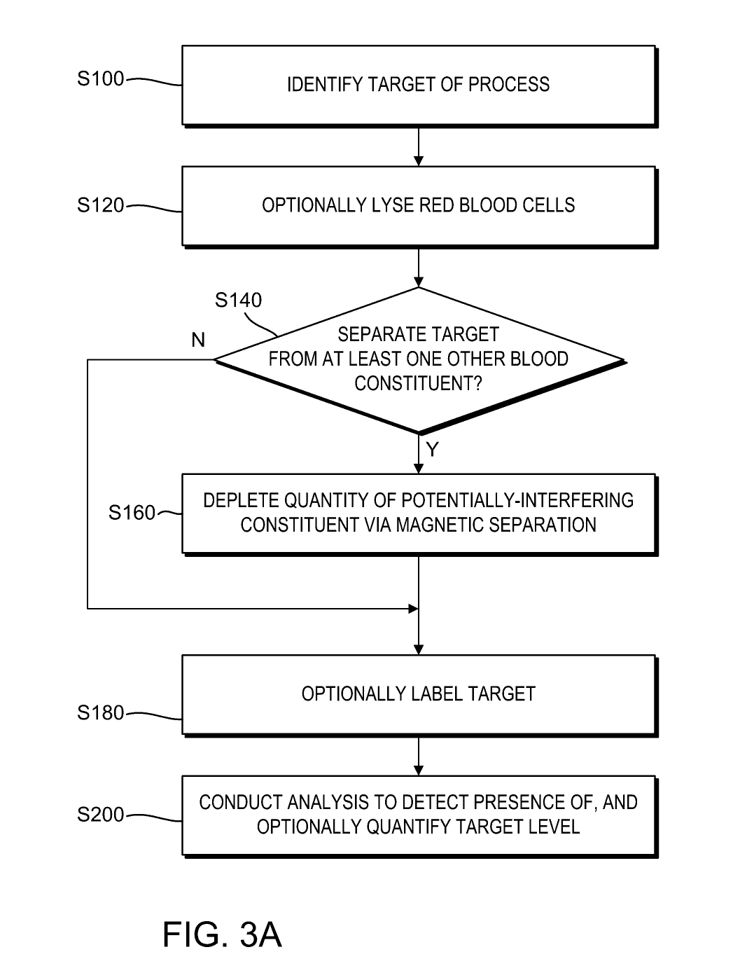

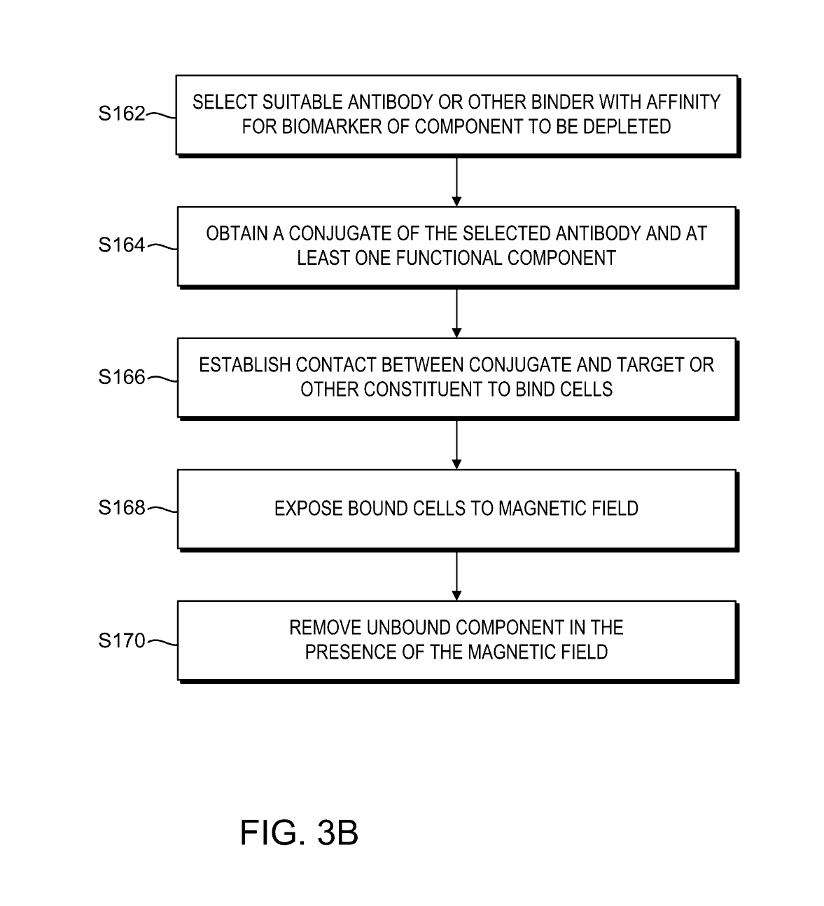

A general overview of an assay method of analyzing a human blood sample to at least detect, and optionally quantify the presence of circulating tumor cells (“CTCs”) according to an illustrative embodiment is depicted by the flow diagram of FIG. 3A. Unless specified otherwise herein, the steps performed do not necessarily have to occur in the order presented, and steps appearing separately can optionally be combined into a single step without departing from the scope of the present disclosure. The specific CTC to be detected as the target is identified at step S100. Because CTCs resulting from different forms of cancer (e.g., ovary, lung, breast, endometrium, kidney, brain, etc.) may express different biomarkers, selection of the CTC of interest at the target will play a significant role in the selection of one or more appropriate antibodies to bind a conjugate to the biomarker(s) of interest as discussed in detail below.

At step 120, the red blood cells can be lysed using any suitable lysing buffer such as ACK Lysing Buffer, from ThermoFisher Scientific, for example. Lysing the red blood cells may simplify, and improve the accuracy of the analysis of flow cytometry results relative the analysis of results for samples that have not had the red blood cells lysed. For other applications, lysis of the red blood cells may offer little, if any benefit, and may be omitted.

If, at step S140, it is determined that at least partial separation of the target from one or more blood constituents that could potentially interfere with the analysis is not advantageous, the assay process can proceed with the labeling of the CTCs or other target in the blood at step S180. If, however, at least partial elimination of a source of potential interference is desired at step S140, depletion of the component desired to be removed can be performed at step S160 via magnetic separation. FIG. 3B shows a flow diagram schematically depicting an illustrative embodiment of a magnetic separation method for separating a component of the blood that could potentially interfere with, or complicate the detection and/or quantification of the CTCs or other target. At step S162, the suitable antibody or other binder with an affinity toward a biomarker expressed by the component to be depleted is selected. A conjugate comprising the selected antibody and a magnetic microparticle can be obtained (e.g., purchased or synthesized) at step S164. The conjugate is then combined with the blood sample and allowed to incubate, at step S162, for a sufficient period of time, with optional agitation, to allow the conjugate to become bound to the component to be depleted. Following incubation, the blood is exposed to a magnetic field of sufficient strength, generated externally of the container in which the blood sample is disposed, to attract the magnetic microparticles, and the component to be depleted, against the walls of the container at step S168. In the presence of the magnetic field holding the magnetic microparticles and the component to be depleted against the container walls, the liquid in the container can be decanted or drawn therefrom at step S170, leaving the component to be depleted behind.

As a specific example, it may be desirable to first deplete the leukocytes present in blood before using flow cytometry or another technology to detect and/or quantify CTCs in human blood. According to the present example anti-human CD45 scFv can be selected at step S162 as a suitable antibody for the CD45 antigen biomarker, which is expressed by leukocytes. It may be desirable to deplete the leukocytes in the blood prior to labeling CTCs with, for example, a conjugate including folic acid or some other ligand antibody, which would facilitate binding of the conjugate to folic acid receptors (FR) on the CTCs. However, since FR are expressed by leukocytes, albeit to a lesser extent than by CTCs, attempting to label CTCs with an anti-folate receptor without first depleting the leukocyte population may complicate or limit the accuracy of the flow cytometry analysis. A conjugate comprising anti-human CD45 scFv and a magnetic microparticle obtained at step S164 can be combined with the blood and incubated to bind the conjugate to the leukocytes at step S166. While the blood container is disposed within a magnetic field at step S168 urging the bound conjugate-leukocytes toward the container wall, the liquid can be drawn from the container with a pipette. This removed liquid will include a much higher ratio of CTCs to leukocytes than the blood sample before magnetic separation.

Although the foregoing example involved binding a magnetic microparticle to the leukocytes to be depleted, alternate embodiments can involve binding a magnetic microparticle to the target of interest using a selective antibody with a higher affinity for the target than the component to be depleted. Thus, rather than removing a portion of the component to be depleted from the liquid, the alternate embodiments can involve removing the target from the liquid, and re-suspending the target in a buffer solution, for example. The end result of a reduced population of the component to be depleted achieved by each embodiment, however, is similar.

Referring once again to FIG. 3A, if the leukocytes removed in the above example are the target of interest, and more than simple isolation is desired, the leukocytes can optionally be labeled at step S180. Labeling the leukocytes can be performed as part of the magnetic separation process that is the subject of FIG. 3B. For example, the functional component of the conjugate obtained at step S164 can optionally include not only the magnetic microparticle, but also a fluorophore. Thus, when the conjugate is bound to the leukocytes at step S166, separated as a result of being exposed to the magnetic field at step S168 and removed at step S170, the isolated leukocytes can be re-suspended in a buffer solution and analyzed using flow cytometry as described below, for example.

The subject of the present analysis will often be the CTCs that remain in the liquid following the magnetic separation to deplete the leukocytes performed at step S160 in FIG. 3A. With the leukocyte population depleted, the CTCs can now be labeled with a conjugate including, for example, an anti-folate receptor or some other ligand antibody, which would facilitate binding of the conjugate to FRs on the CTCs at step S180. The process of labeling the CTCs is similar to the process of using magnetic separation to deplete the leukocytes shown in FIG. 3B. Again, a suitable antibody or other binder with specificity to the biomarker (e.g., folic acid receptors of CTCs) is selected at step S162, and a conjugate comprising the selected antibody and a functional component in the form of a fluorescent-magnetic microparticle is obtained at step S164. The conjugate is combined with the CTC-containing liquid and allowed to incubate at step S166 before the container is exposed to a magnetic field at step S168. In the presence of the magnetic field, the liquid can be decanted, drawn or otherwise removed from the container at step S170, leaving the bound CTCs magnetically attracted to the wall of the container. The bound CTCs can then be re-suspended in a buffer solution before being analyzed using flow cytometry at step S200 of FIG. 3A.

The examples above and discussed hereinafter involve the leukocytes being depleted, followed by the labeling of CTCs with the anti-folate receptor to allow for flow cytometry analysis of the CTC population in human blood. However, it is to be understood that the labeling of CTCs with the anti-folate receptor (or any other antibody for cancerous biomarkers) can optionally be performed without first depleting the leukocyte population. For such embodiments, the antibody selected can exhibit a selectivity specific to the biomarker of interest, without exhibiting a significant affinity for leukocyte markers to an extent that would statistically impact the analysis of flow cytometry results.

Although use of the anti-human CD45 scFv is described throughout the present application as an illustrative embodiment of the antibody exhibiting an affinity for the CD45 antigen, the present disclosure is not so limited. Instead, any suitable molecule having a greater affinity for a biomarker with an expression indicative of a specific condition or the presence of a specific cell sought to be detected can be bonded or otherwise coupled to a fluorescent magnetic microsphere can be used. Accordingly, it is to be understood that the molecule conjugated with the fluorescent magnetic microsphere is to be selected based on the specific biomarker that is the focus of a particular application. Examples of other suitable antibodies include, but are not limited to anti folate receptor antibodies, polyclonal, monoclonal, scFv, aptamer, lectin, peptides, etc. to targets including folic acid receptor, anti-CD45 MoAbCD45, Nodal, DCKL1, etc., and the like. Further, a combination of a plurality of different antibodies can optionally be used concurrently, simultaneously, or in series in an effort to detect and optionally quantify the presence of a plurality of different biomarkers. However, for the sake of brevity and to clearly describe the present technology, many of the specific examples are described herein as utilizing anti CD45 scFv, alone, or in addition to another antibody such as anti-folate receptors, for example, as part of the same assay procedure.

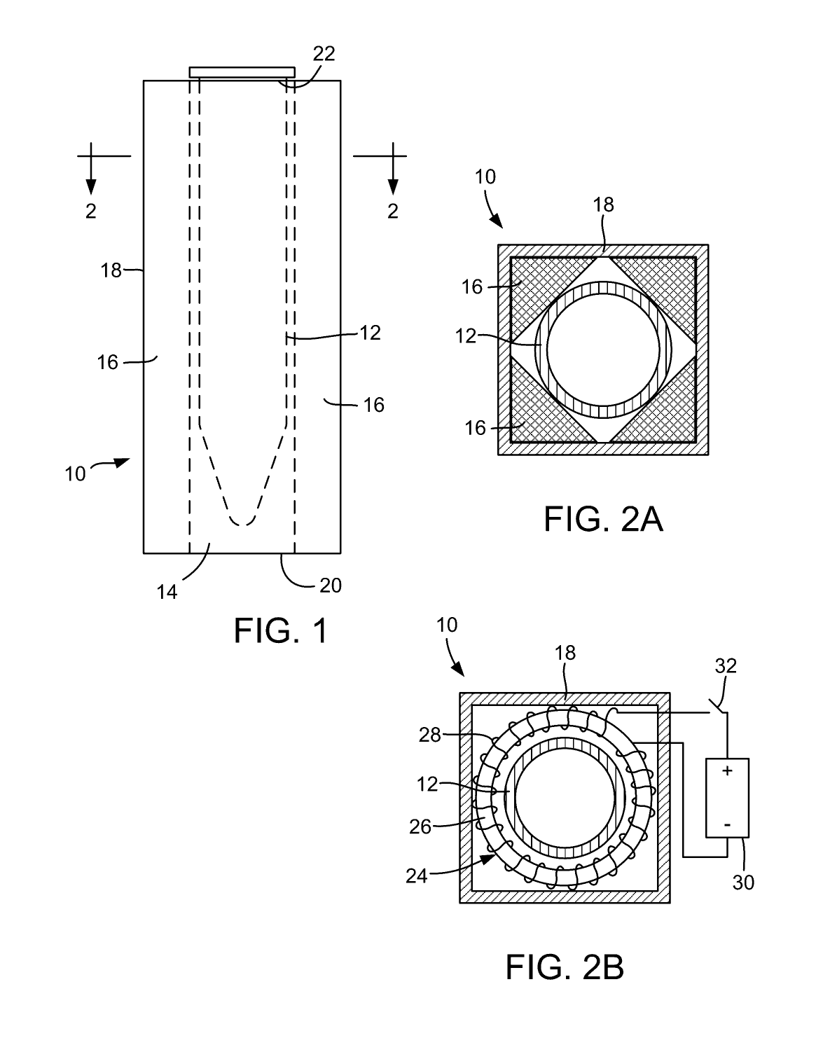

Following incubation of leukocytes with the fluorescent magnetic microspheres the liquid suspension is exposed to a magnetic field generated by a magnetic such as that shown in FIGS. 1 and 2 to facilitate separation of the leukocytes bound and labeled with the fluorescent magnetic microparticles from the liquid medium. As shown in FIG. 1, the suspension is contained within a sample tube 12 received within a space 14 surrounded by a plurality (four in the embodiment appearing in FIGS. 1 and 2) of rare-earth-element-containing magnets 16. Shown clearly in FIG. 2, the magnets 16 are arranged in each corner of a rectangular housing 18, which can be formed from plexiglass, a polymeric material, or any other non-magnetic material. The housing 18 may have a closed base 20 and open top 22 to facilitate insertion of the sample tube 12 into the space 14, surrounded by the four magnets 16 positioned along each of the four vertical sides of the housing form a magnetic field entirely about the sample tube 12. Examples of the sample tube 12 include a culture tube, centrifuge tube, or any suitable container or vessel to hold a suspension.

Depending on the size of the sample tube 12, the volume of contents to be exposed to the magnetic field, or any other factor, the geometry of the magnetic separator 10, the magnets 16, and/or placement of the magnets 16 can be changed for maximum effectiveness. For example, an alternate embodiment of the magnet separator 10 including an electromagnet 24 is shown in FIG. 2B, and includes a housing 18 enclosing a toroidal core 26 about which a coil 28 formed from an electrically-conductive material is wound. The coil 28 is selectively connected to a power supply 30, such as a low voltage (e.g., 12V or less) DC power supply for example, by a switch 32. Embodiments of the switch 32 include solid-state switching devices such as power transistors, magnetically-actuated relays, and the like. Regardless of the specific configuration of the switch 32, operation of the switch 32 can be controlled by a microprocessor-based controller specifically programmed with computer-executable instructions to cause the electromagnet 24 to generate the magnetic field for separating the magnetic microparticles bound to the target cells from the suspension liquid as described herein, and subsequently terminate the magnetic field. Although the toroidal electromagnet 24 is illustrated in FIG. 2B due to the existence of the magnet field primarily inside the space encircled by the toroid, the present disclosure is not so limited. Instead, the present embodiment encompasses any selectively-activated electromagnet that generates, in response to conduction of an electric current, a magnet field suitable to separate the magnetic microparticles bound to the target cells from the suspension liquid.

The magnetic field is applied to the suspension inside the sample tube 12 disposed within the space 14, resulting in the magnetic microparticles bound to the target cells migrating and affixing to the interior wall(s) of the sample tube 12. While the magnetic microparticles bound to the target cells are still subjected to the magnetic field, the supernatant may be decanted, drawn from, or otherwise removed from the sample tube 12, thereby isolating the microparticles anchored to the interior surfaces of the sample tube 12 by the magnetic field. The isolated cells bound to the microparticles may then be washed or re-suspended in a buffer solution, before optionally undergoing further analysis, such as flow cytometry.

The present system and method includes probes that exhibit an affinity for, and bind to a CD45 antigen. Such probes result from the coupling of anti-CD45 scFv, a ligand that binds to the CD45 antigen, with approximately 0.3 μm diameter fluorescent magnetic microspheres, or microspheres of any suitable diameter, shape and/or size to be bound with the particular antibody for targeting the specific biomarker sought to be detected. According to alternate embodiments, the microspheres can optionally have a shape other than spherical, and can have an exterior dimension up to approximately 1 μm, or up to approximately 0.9 μm, or up to approximately 0.8 μm, or up to approximately 0.7 μm, or up to approximately 0.6 μm, or up to approximately 0.5 μm, or up to approximately 0.4 μm, for example. Examples the fluorescent magnetic microspheres include, but are not limited to, embedding type and core shell type polystyrene magnetic beads. Embedding type polystyrene magnetic beads can be formed by embedding magnetic nanoparticles (e.g., having a dimension spanning the particle that is no greater than approximately 100 nm) of Fe₃O₄, or other magnetically-attractive material, in monodisperse polystyrene microspheres. Core type polystyrene magnetic beads can be prepared by coating a thin layer of iron oxide onto polystyrene microspheres (e.g., diameters of approximately 0.5 μm or less). However, alternate embodiments of the magnetic microspheres include, but are not limited to poly(methyl methacrylate (PMMA), poly(lactic-co-glycolic) acid (PLGA), and polycaprolactone (PCL). According to various alternate embodiments, the microspheres may be fluorescent, phosphorescent, color dyed, surface modified, and near-IR responsive. Further, other embodiments can optionally involve labeling components that are magnetic and non-magnetic, which can be utilized if magnetic separation is not desired. The resulting 0.3 μm fluorescent magnetic microspheres derivatized with anti CD45 scFv (0.3 μm-Fl-anti-CD45) provide a convenient probe that may be used to label, identify and isolate CD45-expressing cells. The method may include (a) conjugation of anti-CD45 scFv to 0.3 μm fluorescent magnetic microspheres, and (b) purification of the 0.3 μm-Fl-anti-CD45 conjugate by magnetic separation.

The examples above discuss the coupling of anti-CD45 scFv with approximately 0.3 μm diameter fluorescent magnetic microspheres. However, alternate embodiments involve forming probes designed to bind to the CD45 antigen by derivatizing 0.3 μm diameter fluorescent magnetic microspheres with anti-CD45 monoclonal antibody (MoAb), a ligand which binds to the CD45 antigen. The resulting 0.3 μm fluorescent magnetic microspheres derivatized with anti-CD45 MoAb (0.3 μm-Fl-anti-CD45 MoAb) provide a convenient probe that could be used to label, identify and isolate CD45-expressing cells. An illustrative embodiment of the method may include (a) conjugation of anti-CD45 MoAb to 0.3 μm fluorescent magnetic microspheres, followed by (b) purification of the 0.3 μm-Fl-anti-CD45 MoAb conjugate by magnetic separation.

According to another specific example, leukocytes in whole blood are labeled and isolated using fluorophore-magnetic microsphere-ligand conjugates as the probes, followed by magnetic separation of the leukocytes conjugated to the probes from the suspension liquid. The method may include lysis and removal of the red blood cells (RBCs) before, during or after the labeling procedure, and before the magnetic separation is performed, resulting in an increased fluorescent signal obtained from the labeled leukocytes, and a decreased background signal relative to the signal obtained without first performing the lysis and removal of the RBCs. The resulting increased signal to noise ratio allows greater sensitivity in the detection of labeled cells. In such example, the method may include (a) collection of whole blood sample and lysis of the RBC fraction, (b) labeling of leukocytes by incubation with 0.3 μm fluorescent magnetic microspheres derivatized with anti CD45 scFv, (c) isolation of the labeled leukocytes by magnetic separation, and (d) analysis of the samples by flow cytometry.

It is advantageous to reduce sample processing times during clinical procedures in order to allow the generation of potentially life-saving clinical results in a more-timely manner. In an example, the procedure time may be shortened by combining the red blood cell (RBC) lysis step with the antigen labeling step. For example, human blood may be incubated simultaneously or concurrently with CD45-targeting probes (including 0.3 μm fluorescent magnetic microspheres derivatized with anti-CD45 ligand) and RBC lysis buffer. The resulting CD45-labeled leukocytes may then be isolated by the process of magnetic separation. In other words, the system and method may include (a) simultaneous or concurrent RBC lysis and labeling of leukocytes by incubation with anti CD45 fluorescent magnetic microspheres, (b) isolation of the labeled leukocytes by magnetic separation, and then (c) detection and measurement of the labeled leukocytes by flow cytometry once the isolated and labeled leukocytes are removed from the suspension liquid.

According to alternate embodiments, certain unnecessary reagents and time-consuming steps can optionally be eliminated from the protocol. For example, a single labeling reagent comprising fluorescent magnetic microparticles functionalized with folic acid or some other ligand which would facilitate binding of the labeling reagent to folic acid receptors (FR) on the CTC surface could be utilized. The use of this labeling reagent would allow the direct capture of FR-overexpressing CTCs from the blood sample by the process of magnetic separation, and would eliminate the necessity for the red blood cell (RBC) lysis step and use of a CD45-labeling reagent for depletion of normal leukocytes during sample preparation and analysis, as described elsewhere herein. The method according to the present embodiment would result in lower sample processing costs and rapid sample processing times, thus allowing the generation of potentially life-saving clinical results in a more-timely manner.

According to such an embodiment, a blood sample would be drawn from a patient or test subject. The folic acid-fluorescent magnetic microparticle reagent would be added to the blood sample, and the resulting mixture would be incubated to allow binding of the labeling reagent to any FR-overexpressing CTCs in the sample. The blood sample would then be placed into the magnetic separator 10 during which time the particle-labeled CTCs would be drawn to, and affixed to the interior surface of the sample tube 12 by the magnetic field. Other blood components, such as RBCs, normal leukocytes, platelets, etc., would not be significantly repositioned within the blood by the magnetic field, and would therefore remain in suspension. While the sample remains in the magnetic separator 10 and exposed to the magnetic field, the blood would then be decanted, leaving the labeled CTCs affixed to the interior surface of the sample tube 12. The sample tube 12 would then be removed from the magnetic separator 10 and the isolated CTCs would be washed and/or re-suspended in a buffer solution, and flow count beads, such as Invitrogen™ CountBright™ Absolute Counting Beads or Invitrogen™ AccuCheck Counting Beads from ThermoFisher Scientific, for example, can be added to allow precise quantitation of the CTCs by flow cytometry. The isolated CTCs would then be quantitated by flow cytometry by detection and measurement of the fluorescent dye component of the fluorescent magnetic microparticle labeling reagent bound to the CTCs.

The expected benefits from this modified, rapid, in vitro assay procedure include, but are not limited to: 1.) Lower cost on a per test basis by elimination of the RBC lysis buffer and the anti-CD45 PE-Cy5 labeling reagent used in the current protocol; 2.) Simplification of the procedure by reducing the total number of steps required in the protocol, making the procedure more user friendly; 3.) Simplification of sample analysis by elimination of the need to electronically-deplete (or subtract) the normal, CD45-expressing leukocytes from the processed sample; and 4.) Reduction of sample processing time by elimination of the RBC lysis and CD45-labeling steps and simplification of sample analysis.

### Example 1

### Preparation of CD45 Antigen-Expressing Surrogate Cells

Surrogate CD45 antigen-expressing cells were prepared by derivatizing 6 μm diameter polystyrene micro-particles with human CD45 antigen. The orientation of the CD45 antigen bound to the 6 μm microspheres was such that the CD45 antigen remained immunoaccessible on the surface of the 6 μm microsphere-CD45 constructs (6 μm-CD45 microspheres). The resulting 6 μm-CD45 microspheres provided a convenient CD45 antigen-expressing cell simulant for the screening of probes designed to target and bind to CD45-expressing cells.

A CD45 solution was prepared to a concentration of 5 μg/mL (0.5%) in sodium acetate buffer. CD45 was then conjugated to 6×10⁶ 6 μm diameter polystyrene micro-particles coated with protein A. Protein A is a 42 kDa protein isolated from the cell wall of the bacterium Staphylococcus aureus, which is able to bind immunoglobulins in addition to modifying the surface of the microspheres for covalent coupling of other proteins such as antibodies or antibody fragments (scFv).

The resulting 6 μm-CD45 microsphere conjugate comprised the CD45 antigen-expressing surrogate cells.

### Example 2

### Preparation of Fluorescent Magnetic Microspheres Conjugated to Anti-CD45 scFv

Probes designed to bind to the CD45 antigen were prepared by derivatizing 0.3 μm diameter fluorescent magnetic microspheres with anti-CD45 scFv, a ligand which binds to the CD45 antigen. The resulting 0.3 μm fluorescent magnetic microspheres derivatized with anti CD45 scFv (0.3 μm-Fl-anti-CD45 scFv) provided a convenient probe that could be used to label, identify and isolate CD45-expressing cells. All scFv coated magnetic polystyrene microparticles were purchased from Spherotech, an ISO 9001:2008 registered company located in Lake Forest, Ill. The example involved (a) conjugation of anti-CD45 scFv to 6 μm fluorescent magnetic microspheres, (b) purification of the 0.3 μm-Fl-anti-CD45 conjugate by magnetic separation.

Three anti-CD45 scFv solutions (0.1 M phosphate buffered saline, pH 7.0) were prepared to concentrations of 2.39 μg/mL, 1.62 μg/mL and 0.87 μg/mL [A 50 μL volume of each of the three anti-CD45 scFv solutions was added to 3 separate 0.2 mL volumes of acetate buffer containing EDC and 1 mL of 0.3 micron fluorescent magnetic micro-particles containing 4.4×10⁹ beads. Final anti-CD45 scFv coating concentrations were 0.10 μM, 0.06 μM and 0.03 μM respectively. The resulting bead coating suspensions were then incubated for 3 hours at room temperature.

Following the 3 hour incubation, the resulting 0.3 μm-Fl-anti-CD45 conjugates were magnetically separated and washed with phosphate buffered saline, pH=7.4 (PBS). Isolation by magnetic separation was repeated, and the purified 0.3 μm-Fl-anti-CD45 conjugates were resuspended in fresh PBS to a final concentration of 4.4×10⁹ beads/200 μL.

### Example 3

### Preparation of Fluorescent Magnetic Microspheres Conjugated to Anti-CD45 Monoclonal Antibody

Probes designed to bind to the CD45 antigen were prepared by derivatizing 0.3 μm diameter fluorescent magnetic microspheres with anti-CD45 monoclonal antibody (MoAb), a ligand which binds to the CD45 antigen. The resulting 0.3 μm fluorescent magnetic microspheres derivatized with anti-CD45 MoAb (0.3 μm-Fl-anti-CD45 MoAb) provided a convenient probe which could be used to label, identify and isolate CD45-expressing cells. The example involved (a) conjugation of anti-CD45 MoAb to 0.3 μm fluorescent magnetic microspheres, (b) purification of the 0.3 μm-Fl-anti-CD45 MoAb conjugate by magnetic separation.

A 50 μL volume of a 25 μg/mL monoclonal anti-CD45 solution (BD Catalog number 347460 was mixed with 0.2 mL of 0.1 M acetate buffer, pH 5.0 containing 2 mM EDC (1-ethyl-3-(3-dimethylaminopropyl) carbodiimide hydrochloride and 1.0 mL of 0.3 micron fluorescent magnetic micro-particles (4.4×10⁹ beads).

The resulting monoclonal anti-CD45 coating concentration was 0.17 μM. The resulting bead coating suspension was then incubated for 3 hours.

Following the 3 hour incubation, the resulting 0.3 μm-Fl-anti-CD45 MoAb conjugate was magnetically separated and washed with phosphate buffered saline, pH=7.4 (PBS). Isolation by magnetic separation was repeated, and the purified 0.3 μm-Fl-anti-CD45 MoAb conjugates were resuspended in fresh PBS to a final concentration of 4.4×10⁹ beads/200 μL. The magnetic separators were custom-made and are not commercially available.

### Example 4

### Demonstration of Migration of Magnetic Microspheres Through Biological Fluid Simulant

The ability of 0.3 μm fluorescent magnetic microspheres (microparticles) derivatized with anti CD45 scFv (see Example 2 above) to migrate through, and be recovered from, viscous biological fluids under the influence of a magnetic field was demonstrated in a biological fluid simulant. The 0.3 μm-anti-CD45 particles were suspended in 1% milk, which served as a biological fluid simulant, and were then separated and recovered from the fluid by use of a magnetic separator device. The example involved (a) preparation of the biological fluid simulant/microparticle suspension, (b) magnetic separation and isolation of the magnetic microparticles, and (c) detection of the magnetic microparticles.

1% cow milk was selected for use as a biological fluid simulant, and was used undiluted. The viscosity of 1% milk at room temperature (20° C.) is approximately 1.5 centipoise (cP). This compares to the viscosities of human serum and plasma, having viscosities of 1.4 cP and 1.65 cP, respectively. (Bakshi 1984, Momen-Heravi, 2012).

Into a 15 mL conical bottom, polypropylene [standard commercially available] centrifuge tube, 1.0 mL of biological fluid simulant (1% milk) was placed. To the biological fluid simulant was then added 1 μL of 0.3 μm-anti-CD45 particles (particle density of 0.3 μm-anti-CD45 stock was 7×10⁵ particles/μL). The tube was gently mixed by vortexing. The resulting suspension constituted the biological fluid simulant/microparticle suspension. Two identical biological fluid simulant/microparticle suspensions were prepared in this way.

The tubes containing the biological fluid simulant/microparticle suspensions were placed into a magnetic separator to allow the 0.3 μm-anti-CD45 particles to be drawn and affixed to the interior sides of the centrifuge tube. One tube was allowed to remain in the magnetic separator for 5 minutes, while the second tube remained in the magnetic separator for 10 minutes. However, residence times within the magnetic separator can be of any suitable duration, such as at least three (3 min.) minutes. In this way, a comparison of the effect of incubation time on separation efficiency was made. Following magnetic separation, each tube was left in the magnetic separator, and the supernatant in each tube was gently decanted. The contents of each tube was then gently washed using 1 mL of PBS, and finally re-suspended in a volume of 1.0 mL fresh PBS.

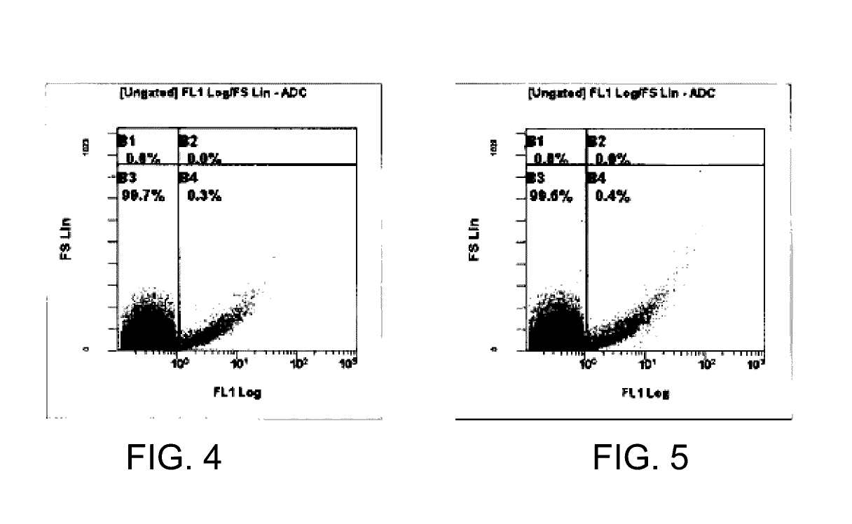

The resulting 2 samples were analyzed for fluorescent particle content by flow cytometry, Beckman-Coulter FC500 with a 488 Argon Laser. A high flow rate was used, and a total of 1,000,000 events were recorded during measurement. The results of the flow cytometry analysis are shown in FIGS. 4 and 5.

The results demonstrate the migration, isolation and recovery of the 0.3 μm-anti-CD45 particles from the biological fluid simulant. Comparing the 10 minute magnetic separation time (FIG. 5) to the 5 minute separation time (FIG. 4), a 23.6% increase in particle counts was noted for the longer separation time (3036 vs. 3752 events, respectively, B4 quadrant). This result suggests that a magnetic separation time of greater than 5 minutes may be required for the separation to reach completion.

### Example 5

### Demonstration of Binding of Fluorescent Magnetic Microspheres Conjugated with Anti-CD45 scFv to a CD45 Antigen Target

Surrogate CD45 antigen-expressing cells, comprising 6 μm polystyrene microspheres derivatized with human CD45 antigen (6 μm-CD45 microspheres), were suspended in phosphate buffered saline, pH=7.4 (PBS) to a concentration of 2×10⁶ particles/mL. This suspension served as the 6 μm-CD45 microspheres working suspension. Three separate lots of CD45-targeting probes comprising 0.3 μm fluorescent magnetic microspheres derivatized with anti CD45 scFv (0.3 μm-Fl-anti-CD45 microspheres) were evaluated:

Lot A: 2.39 μg/mL

Lot B: 1.62 μg/mL

Lot C: 0.87 μg/mL

The stock concentration of all 3 lots of 0.3 μm-FL-anti-CD45 microspheres was 7×10⁵ particles/μL. Labeling mixtures were prepared in each of 6, 12×75 mm, polystyrene tubes by adding the following reagents to each tube:
Sample 1: 0.5 mL PBS only
Sample 2: 0.5 mL 6 μm-CD45 microspheres working susp′n+14.3 μL PBS
Sample 3A: 0.5 mL 6 μm-CD45 microspheres working susp′n+14.3 μL 0.3 μm-Fl-anti-CD45 microspheres (Lot A)
Sample 4B: 0.5 mL 6 μm-CD45 microspheres working susp′n+14.3 μL 0.3 μm-Fl-anti-CD45 microspheres (Lot B)
Sample 5C: 0.5 mL 6 μm-CD45 microspheres working susp′n+14.3 μL 0.3 μm-Fl-anti-CD45 microspheres (Lot C)

Sample 6: 14.3 μL 0.3 μm-Fl-anti-CD45 microspheres (Lot C)+0.5 mL PBS

Each tube was mixed by gentle vortexing. Table 1 summarizes the test parameters for each sample.

TABLE 1

| | | No. of 0.3 μm | | |
|---|---|---|---|---|
| Sample | | microspheres/ | No. of 6 μm | Ratio |
| No. | Test Mixture | tube | microspheres/tube | (0.3 μm:6 μm) |
| 1 | PBS Control | 0 | 0 | NA |
| 2 | 6 μm-CD45 microspheres only | 0 | 1 × 10⁶ | NA |
| | (control) | | | |
| 3A | 6 μm-CD45 microspheres + 0.3 μm- | 1 × 10⁷ | 1 × 10⁶ | 10:1 |
| | Fl-anti-CD45 microspheres (Lot A) | | | |
| 4B | 6 μm-CD45 microspheres + 0.3 μm- | 1 × 10⁷ | 1 × 10⁶ | 10:1 |
| | Fl-anti-CD45 microspheres (Lot B) | | | |
| 5C | 6 μm-CD45 microspheres + 0.3 μm- | 1 × 10⁷ | 1 × 10⁶ | 10:1 |
| | Fl-anti-CD45 microspheres (Lot C) | | | |
| 6 | 0.3 μm-Fl-anti-CD45 microspheres | 1 × 10⁷ | 0 | NA |
| | (Lot C) only (control) | | | |

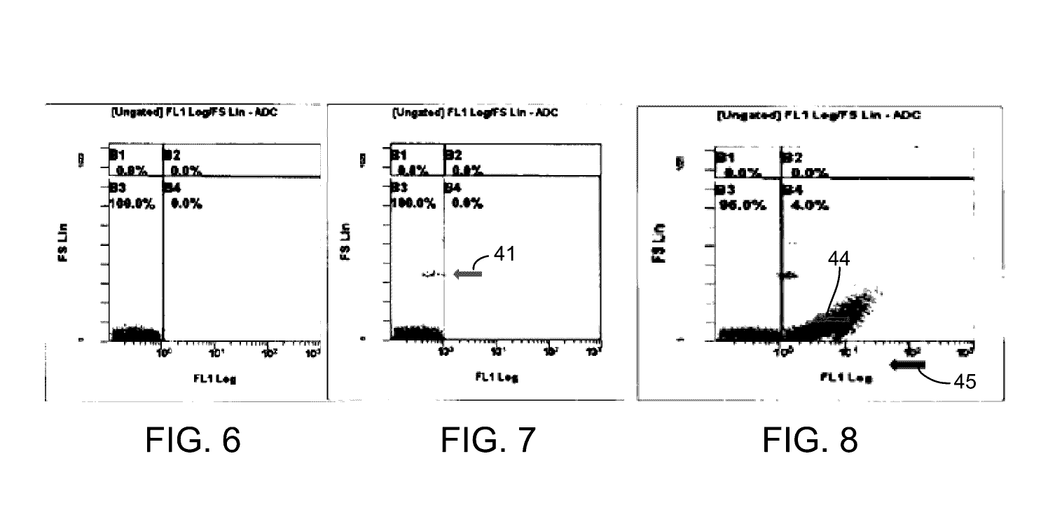

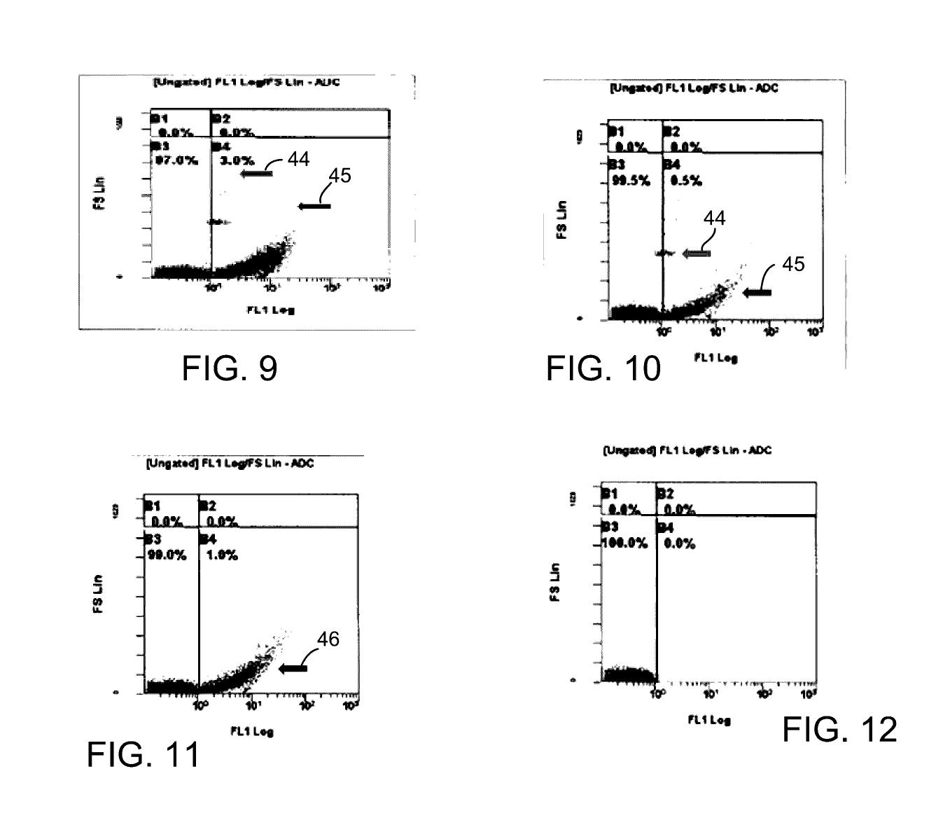

The resulting 6 samples were analyzed for fluorescent labeling density by flow cytometry. The results of the flow cytometry analysis are shown in FIGS. 6-11.

FIG. 6, corresponding to Sample A (the PBS control), shows a clean background with no signal. FIG. 7 shows the unlabeled 6 μm-CD45 microspheres as visualized by light scattering (blue arrow 41). FIGS. 8-10, show results obtained from the mixture of 6 μm-CD45 and 0.3 μm-anti-CD45 (Lots A, B and C, respectively), and demonstrate fluorescent labeling of the Surrogate CD45 antigen-expressing cells (6 μm-CD45 microspheres) by 0.3 μm-Fl-anti-CD45 microspheres (green arrows 44), and also show unbound 0.3 μm-Fl-anti-CD45 microspheres (red arrows 45). FIG. 11 shows unbound 0.3 μm-Fl-anti-CD45 microspheres (red arrows 46) only.

Each of the three lots of 0.3 μm-Fl-anti-CD45 microspheres (Lot A, Lot B and Lot C) successfully labeled surrogate CD45 antigen-expressing cells with levels of fluorescence intensity clearly above background.

### Example 6

### Demonstration of Density-Dependent Binding of Fluorescent Magnetic Microspheres Conjugated with Anti-CD45 scFv to a CD45 Antigen Target

The binding of 0.3 μm fluorescent magnetic microspheres derivatized with anti CD45 scFv (0.3 μm-Fl-anti-CD45, see Example 2 above) to surrogate CD45 antigen-expressing cells, comprising 6 μm polystyrene microspheres derivatized with human CD45 antigen (6 μm-CD45, see Example 1 above) was shown to occur in a density-dependent manner. A fixed number of 6 μm-CD45 were incubated with increasing numbers of 0.3 μm-Fl-anti-CD45, and the relationship between 0.3 μm-Fl-anti-CD45 concentration and binging density was determined by flow cytometry. The example involved (a) labeling a fixed number of surrogate CD45 antigen-expressing cells with increasing numbers of 0.3 μm-Fl-anti-CD45, and (b) measurement of the labeled surrogate CD45 antigen-expressing cells by flow cytometry.

A 6 μm-CD45 (surrogate CD45 antigen-expressing cells) suspension was prepared to a density of 5×10⁶ particles/mL in phosphate buffered saline, pH=7.4 (PBS). A 0.3 μm-Fl-anti-CD45 stock suspension containing 7×10⁸ particles/mL was used undiluted. Labeling mixtures were prepared in each of 8, 12×75 mm, polystyrene tubes by adding the following reagents to each tube:

Sample 1: 0.5 mL PBS only
Sample 2: 0.5 mL 6 μm-CD45+35.7 μL PBS
Sample 3: 0.5 mL 6 μm-CD45+2.2 μL 0.3 μm-Fl-anti-CD45
Sample 4: 0.5 mL 6 μm-CD45+4.5 μL 0.3 μm-Fl-anti-CD45
Sample 5: 0.5 mL 6 μm-CD45+8.9 μL 0.3 μm-Fl-anti-CD45
Sample 6: 0.5 mL 6 μm-CD45+17.9 μL 0.3 μm-Fl-anti-CD45
Sample 7: 0.5 mL 6 μm-CD45+35.7 μL 0.3 μm-Fl-anti-CD45
Sample 8: 0.5 mL PBS+35.7 μL 0.3 μm-Fl-anti-CD45

Each tube was mixed by gentle vortexing. Table 2 summarizes the test parameters for each sample.

TABLE 2

| Sample | | No. of 0.3 μm | No. of 6 μm | Ratio |
|---|---|---|---|---|
| No. | Test Mixture | microspheres/tube | microspheres/tube | (0.3 μm:6 μm) |
| 1 | PBS Blank | 0 | 0 | NA |
| 2 | 6 μm-CD45 only (control) | 0 | 2.5 × 10⁶ | NA |
| 3 | 6 μm-CD45 + 0.3 μm-Fl-anti-CD45 | 1.56 × 10⁶ | 2.5 × 10⁶ | 0.625:1 |
| 4 | 6 μm-CD45 + 0.3 μm-Fl-anti-CD45 | 3.125 × 10⁶ | 2.5 × 10⁶ | 1.25:1 |
| 5 | 6 μm-CD45 + 0.3 μm-Fl-anti-CD45 | 6.25 × 10⁶ | 2.5 × 10⁶ | 2.5:1 |
| 6 | 6 μm-CD45 + 0.3 μm-Fl-anti-CD45 | 1.25 × 10⁷ | 2.5 × 10⁶ | 5:1 |
| 7 | 6 μm-CD45 + 0.3 μm-Fl-anti-CD45 | 2.5 × 10⁷ | 2.5 × 10⁶ | 10:1 |
| 8 | 0.3 μm-Fl-anti-CD45 only (control) | 2.5 × 10⁷ | 0 | NA |

The resulting 8 samples were analyzed for fluorescein labeling density by flow cytometry.

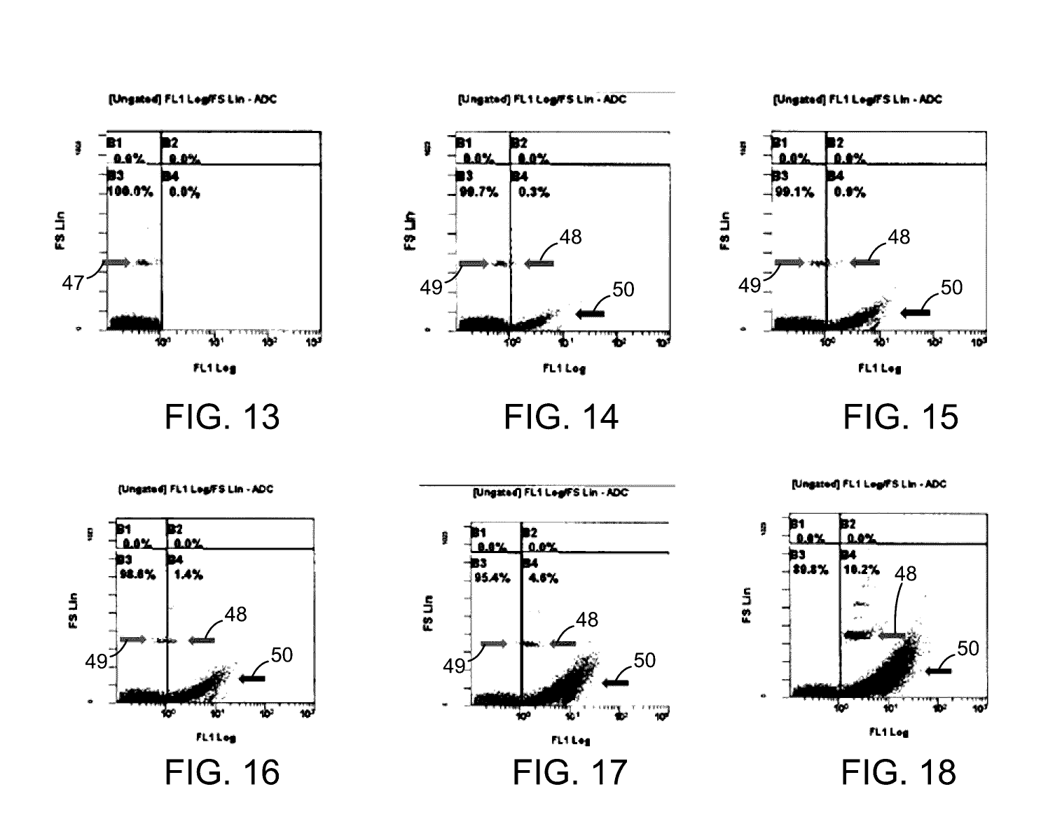

FIG. 12, the PBS blank, shows a clean background with no signal. FIG. 13, 6 μm-CD45 microspheres alone, shows the unlabeled 6 μm-CD45 microspheres as visualized by light scattering (blue arrow 47).

FIGS. 14-18 show fluorescent labeling of the surrogate CD45 antigen-expressing cells (6 μm-CD45) at increasing levels as the number of 0.3 μm-Fl-anti-CD45 particles in the mixture increase (green arrows 48). The amount of unlabeled 6 μm-CD45 (blue arrows 49) was found to decrease as the number of 0.3 μm-Fl-anti-CD45 particles in the mixture increased. Unbound 0.3 μm-Fl-anti-CD45 (red arrows 50) also increased as the ratio of 0.3 μm-Fl-anti-CD45: 6 μm-CD45 increased.

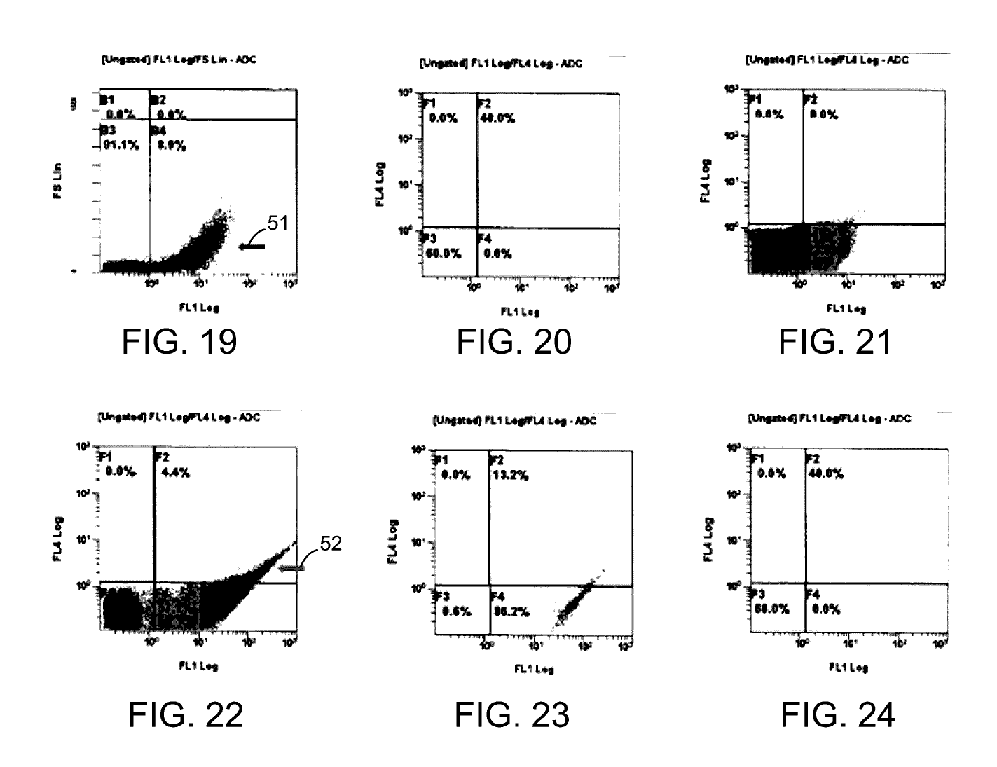

FIG. 19, 0.3 μm-Fl-anti-CD45 microspheres alone, shows unbound 0.3 μm-Fl-anti-CD45 microspheres (red arrows 51) only, at a density of 5×10⁷ particles/mL.

The results of Example 6 show a direct correlation between the density of 0.3 μm-Fl-anti-CD45 and the amount of binding of those particles to the CD45 antigen target on the surrogate CD45 antigen-expressing cells (6 μm-CD45), and, therefore, demonstrate that the binding of 0.3 μm-Fl-anti-CD45 to surrogate CD45 antigen-expressing cells (6 μm-CD45) occurs in a density-dependent manner.

### Example 7

### Demonstration of Binding Fluorescent Magnetic Microspheres to the CD45 Antigen on Viable Human Leukocytes in Whole Blood and Isolation of Labeled Cells by Magnetic Separation

The binding of 0.3 μm fluorescent magnetic microspheres derivatized with anti CD45 scFv (0.3 μm-Fl-anti-CD45, see Example 2 above) to viable human leukocytes in whole blood followed by isolation and recovery of the labeled leukocytes by magnetic separation was demonstrated. The leukocyte fraction obtained from whole human blood was incubated with 0.3 μm-Fl-anti-CD45 magnetic microspheres. The resulting 0.3 μm-Fl-anti-CD45-labeled leukocytes were then isolated by the process of magnetic separation. The example involved (a) labeling of leukocytes by incubation with 0.3 μm fluoresceinated magnetic microspheres derivatized with anti CD45 scFV, (b) isolation of the labeled leukocytes by magnetic separation, and (c) measurement of the labeled leukocytes by flow cytometry.

Whole blood was drawn from a healthy human volunteer and collected in Cell Save® preservative tubes (Clinical Research Solutions). Into each of two 15 mL conical bottom, polypropylene, centrifuge tubes, 0.5 mL of blood was placed. To each tube was then added 1.5 mL of red blood cell (RBC) lysis buffer (G-Biosciences) and the tube contents were mixed by gentle vortexing. Following incubation at room temperature for 15 minutes, the tubes were centrifuged at 100×G for 10 minutes at room temperature. Supernatants were decanted and each of the two resulting leukocyte pellets were resuspended in 0.5 mL of phosphate buffered saline, pH=7.4 (PBS). Based on the normal clinical range for human blood, each leukocyte suspension was considered to contain 7×10⁶ cells/mL, or 3.5×10⁶ cells/tube. To one of the leukocyte suspensions was added 25 μL of 0.3 μm-Fl-anti-CD45 which contained 7×10⁸ particles/mL (1.75×10⁷ particles total). The ratio of 0.3 μm-Fl-anti-CD45 particles per cell was 5:1. The tube contents were mixed by gentle vortexing. The second tube was designated as the unlabeled control and contained leukocytes only. Both tubes were incubated at room temperature for 10 minutes to allow binding of the 0.3 μm-Fl-anti-CD45 to the leukocytes in the first tube. Two additional control groups were also prepared: 1.) PBS blank comprising 0.5 mL of PBS only, and 2.) 0.3 μm particle control comprising 0.5 mL of PBS into which 25 μL of 0.3 μm-Fl-anti-CD45 (7×10⁸ particles/mL) was added. Table 3 summarizes the test parameters for each sample.

TABLE 3

| | No. of 0.3 μm | | |
|---|---|---|---|
| | 0.3 μm-Fl-anti- | No. of | Ratio (0.3 μm |
| Test Mixture | CD45/tube | Leukocytes/tube | particles:leukocytes) |
| PBS Blank (no cells, no label) | 0 | 0 | NA |
| Leukocytes only (unlabeled control) | 0 | 3.5 × 10⁶ | NA |
| Leukocytes + 0.3 μm-Fl-anti-CD45 + | 1.75 × 10⁷ | 3.5 × 10⁶ | 5:1 |
| mag. separation | | | |
| 0.3 μm-Fl-anti-CD45 + mag. | 1.75 × 10⁷ | 0 | NA |
| separation (0.3 μm particle control) | | | |

The 15 mL conical bottom centrifuge tubes containing the 0.3 μm-Fl-anti-CD45-labeled leukocytes, and the 0.3 μm-Fl-anti-CD45 particle control were placed into magnetic separators for 10 minutes to allow the labeled cells and/or unbound 0.3 μm-Fl-anti-CD45 to be drawn and affixed to the interior sides of the centrifuge tubes. The tubes containing the PBS blank and unlabeled control were set aside. Leaving the tubes in the magnetic separators, the supernatants were decanted in order to remove any unbound material. The tubes containing bound labeled cells and the 0.3 μm-Fl-anti-CD45 particle control were removed from the magnetic separators and the contents of each tube were resuspended in 0.5 mL of PBS.

The resulting 4 samples were analyzed for fluorescent labeling density by flow cytometry.

FIGS. 20-23 show the flow cytometry plots resulting from the 4 samples. FIG. 20, the PBS blank, shows a clean background with no signal. FIG. 21 shows the unlabeled control cells as detected by light scattering. FIG. 22 shows the fluorescent labeling of the human leukocytes by the 0.3 μm-Fl-anti-CD45 probes (green arrow 52). FIG. 23, the particle control, shows the fluorescent signal obtained from unbound 0.3 μm-Fl-anti-CD45 particles in the absence of cells.

The results from Example 7 demonstrate binding of the 0.3 μm-Fl-anti-CD45 probes to the CD45 antigen targets on human leukocytes, and also the isolation and recovery of the labeled cells by the process of magnetic separation.

### Example 8

### Demonstration of the Utility of Red Blood Cell Lysis and Removal to Improve the Signal to Noise Ratio in Suspensions Comprising Cells Labeled with Fluorophore-Magnetic-Microsphere-Ligand Complexes

Example 8, in which leukocytes in whole blood underwent labeling and isolation using fluorophore-magnetic microsphere-ligand conjugates and magnetic separation, demonstrates that lysis and removal of the red blood cells (RBCs) during the labeling procedure resulted in an increased fluorescent signal obtained from the labeled leukocytes, and a decreased background signal. The resulting increased signal to noise ratio allows greater sensitivity in the detection of labeled cells. The example involved (a) collection of whole blood sample and lysis of the RBC fraction, (b) labeling of leukocytes by incubation with 0.3 μm fluorescent magnetic microspheres derivatized with anti CD45 scFv, (c) isolation of the labeled leukocytes by magnetic separation, and (d) analysis of the samples by flow cytometry.

Whole blood was drawn from a healthy human volunteer and collected in Cell Save® preservative tubes (Clinical Research Solutions). Into each of three 15 mL conical bottom, polypropylene, centrifuge tubes, 0.5 mL of blood was placed. One of the three tubes was set aside to serve as the unlysed control. To the other two tubes was then added 1.5 mL of red blood cell (RBC) lysis buffer (G-Biosciences) and the tube contents were mixed by gentle vortexing. The tubes were incubated at room temperature for 15 minutes to allow RBC lysis to occur. The 2 tubes were then centrifuged at 100×G for 10 minutes at room temperature. Supernatants were decanted to remove RBC debris, and each of the 2 resulting leukocyte pellets were resuspended in 0.5 mL of phosphate buffered saline, pH=7.4 (PBS). Based on the known clinical range for human blood, each leukocyte suspension, and the untreated whole blood, was considered to contain 7×10⁶ cells/mL, or 3.5×10⁶ cells/tube.

To one of the leukocyte suspensions, to the tube containing whole blood (unlysed), and to a third tube containing only 0.5 mL PBS, was added 25 μL of 0.3 μm-Fl-anti-CD45 which contained 7×10⁸ particles/mL (1.75×10⁷ particles total). The ratio of 0.3 μm-Fl-anti-CD45 particles per leukocyte was 5:1. The tube contents were mixed by gentle vortexing. A fifth tube was prepared as a PBS blank, and contained 0.5 mL of PBS only. The parameters for each sample are summarized as follows:

| | | No. of 0.3 μm | | |
|---|---|---|---|---|
| Sample | | 0.3 μm-Fl-anti- | No. of | Ratio (0.3 μm |
| No. | Test Mixture | CD45/tube | Leukocytes/tube | particles:leukocytes) |
| 1 | PBS Blank (no cells, no label) | 0 | 0 | |
| 2 | Blood only, w/RBC lysis and | 0 | 3.5 × 10⁶ | NA |
| | wash of WBCs | | | |
| 3 | Blood + 0.3 μm/anti-CD45, | 1.75 × 10⁷ | 3.5 × 10⁶ | 5:1 |
| | w/RBC lysis + mag. | | | |
| 4 | Blood + 0.3 μm/anti-CD45, | 1.75 × 10⁷ | 3.5 × 10⁶ | 5:1 |
| | w/o RBC lysis + mag. | | | |
| 5 | 0.3 μm-Fl-anti-CD45 + mag. | 1.75 × 10⁷ | 0 | NA |
| | separation (0.3 μm particle | | | |

Samples were incubated at room temperature for 10 minutes to allow binding of the 0.3 μm-Fl-anti-CD45 to the leukocytes in samples 3 and 4.

The 15 mL conical bottom centrifuge tubes containing the 0.3 μm-Fl-anti-CD45-labeled leukocytes, and the 0.3 μm-Fl-anti-CD45 particle control (groups 3, 4, 5) were placed into a magnetic separator for 10 minutes to allow the labeled cells and/or unbound 0.3 μm-Fl-anti-CD45 to be drawn and affixed to the interior sides of the centrifuge tubes. The tubes containing the PBS blank and leukocytes only (groups 1 and 2) were set aside. While leaving the tubes in the magnetic separators, the supernatants were decanted in order to remove any unbound material. The tubes containing bound labeled cells and the 0.3 μm-Fl-anti-CD45 particle control were removed from the magnetic separators and the contents of each tube were re-suspended in 0.5 mL of PBS.

Prior to analysis, the 5 resulting samples were transferred to separate 12×75 mm, polystyrene, snap-cap tubes. The 5 samples were analyzed for fluorescein labeling density by flow cytometry. The results of the flow cytometry analysis are shown in FIGS. 24-28.

FIG. 24, the PBS blank, shows a clean background with no signal.

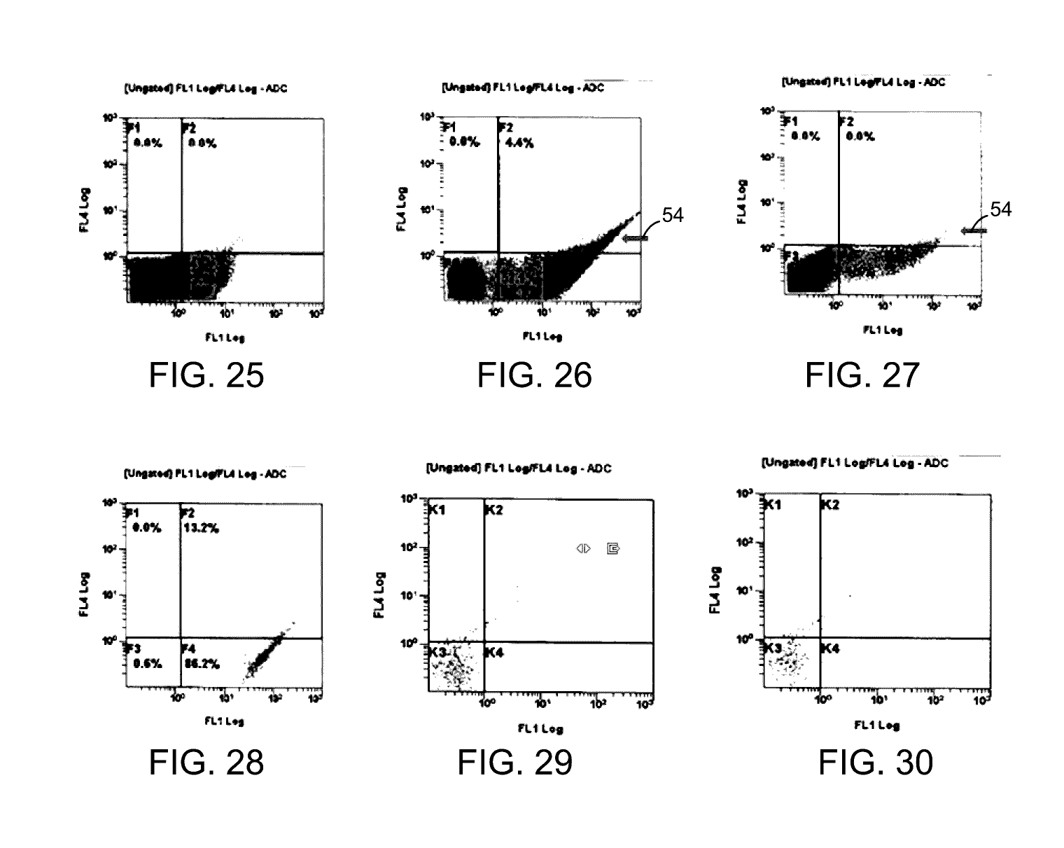

FIG. 25 shows the unlabeled control leukocytes as detected by light scattering.

FIGS. 26 and 27 show fluorescent labeling of the human leukocytes by the 0.3 μm-Fl-anti-CD45 probes (green arrows 54) with and without RBC lysis, respectively. Note the increased fluorescent signal in sample 3, which included the RBC lysis step (FIG. 26) as compared to group 4, which did not include RBC lysis (FIG. 27).

For comparison purposes, FIG. 28, the particle control, shows the fluorescent signal obtained from the 0.3 μm-Fl-anti-CD45 particles in the absence of cells, and confirms that the magnetic separation step was successful.

The results of Example 8 demonstrate the benefit of red blood cell lysis and removal for improvement of signal to noise ratio in suspensions comprising cells labeled with fluorophore-magnetic microsphere-ligand conjugates.

### Example 9

### Shortened Labeling Procedure for the Binding of Fluorescent Magnetic Microspheres TO the CD45 Antigen on Viable Human Leukocytes in Whole Blood, and Isolation of Labeled Cells by Magnetic Separation

It is advantageous to reduce sample processing times during clinical procedures in order to allow the generation of potentially life-saving clinical results in a more-timely manner. In comparison to the procedures described in Examples 7 and 8 above, it was found that procedure time could be shortened by combining the red blood cell (RBC) lysis step with the antigen labeling step. Human blood was incubated simultaneously with CD45-targeting probes (comprising 0.3 μm fluorescent magnetic microspheres derivatized with anti-CD45 ligand) and RBC lysis buffer. The resulting CD45-labeled leukocytes were then isolated by the process of magnetic separation. Example 9 involved (a) simultaneous RBC lysis and labeling of leukocytes by incubation with anti CD45 fluorescent magnetic microspheres, (b) isolation of the labeled leukocytes by magnetic separation, and (c) detection and measurement of the labeled leukocytes by flow cytometry.

Whole blood was drawn from a healthy human volunteer and collected in CELL SAVE® preservative tubes (Clinical Research Solutions). Into each of five 15 mL conical bottom, polypropylene, centrifuge tubes, 1.0 mL of blood was placed. To serve as cell-free particle controls, 1.0 mL of phosphate buffered saline, pH=7.4 (PBS) was placed into each of two additional 15 mL tubes (no blood). Into each of the 7 tubes was then placed 3.0 mL of red blood cell (RBC) lysis buffer (G-Biosciences).

Three separate lots of CD45-targeting probes comprising 0.3 μm fluorescent magnetic microspheres derivatized with anti CD45 scFv (0.3 μm-Fl-anti-CD45 scFv, see Example 2 above) were evaluated:

Lot A: 2.39 μg/mL scFv coating concentration

Lot B: 1.62 μg/mL scFv coating concentration

Lot C: 0.87 μg/mL scFv coating concentration

The concentration of the three 0.3 μm-Fl-anti-CD45 scFv working solutions was 4.4×10⁹ particles/200 μL.

The CD45-specific label comprised 0.3 μm fluorescent magnetic microspheres derivatized with anti-CD45 monoclonal antibody (0.3 μm-Fl-anti-CD45 MoAb, see Example 3 above). The concentration of the 0.3 μm-Fl-anti-CD45 MoAb working solutions was 4.4×10⁹ particles/200 μL.

TABLE 4

| Sam- | | | RBC | | | |
|---|---|---|---|---|---|---|
| ple | | | Lysis | Magnetic | | No. of |
| No. | Blood | PBS | Buffer | Particles | Particle | Particles |
| 1 | 0 mL | 1 mL | 3 mL | 1 μL | 0.3 μm-Fl-anti- | 2.2 × 10⁷ |
| | | | | | CD45 scFv, | |
| | | | | | Lot C | |
| 2 | 0 mL | 1 mL | 3 mL | 1 μL | 0.3 μm-Fl-anti- | 2.2 × 10⁷ |
| | | | | | CD45 MoAb | |
| 3 | 1 mL | 0 mL | 3 mL | 1 μL | 0.3 μm-Fl-anti- | 2.2 × 10⁷ |
| | | | | | CD45 MoAb | |
| 4 | 1 mL | 0 mL | 3 mL | 1 μL | 0.3 μm-Fl-anti- | 2.2 × 10⁷ |
| | | | | | CD45 scFv, | |
| | | | | | Lot A | |
| 5 | 1 mL | 0 mL | 3 mL | 1 μL | 0.3 μm-Fl-anti- | 2.2 × 10⁷ |
| | | | | | CD45 scFv, | |
| | | | | | Lot B | |
| 6 | 1 mL | 0 mL | 3 mL | 1 μL | 0.3 μm-Fl-anti- | 2.2 × 10⁷ |
| | | | | | CD45 scFv, | |
| | | | | | Lot C | |
| 7 | 1 mL | 0 mL | 3 mL | 5 μL | 0.3 μm-Fl-anti- | 1.1 × 10⁸ |
| | | | | | CD45 scFv, | |
| | | | | | Lot C | |

The contents of each sample were mixed by gentle vortexing. To allow binding of the anti-CD45 probes to CD45-expressing leukocytes, and completion of RBC lysis, the resulting mixtures were incubated at room temperature for 60 minutes with gentle vortexing every 15 minutes.

The 15 mL conical bottom centrifuge tubes containing each of the 7 mixtures were placed into magnetic separators for 5 minutes to allow the labeled cells to be drawn and affixed to the interior sides of the centrifuge tubes. While leaving the tubes in the magnetic separators, the supernatants were gently decanted in order to remove any unbound material. The tubes were then removed from the magnetic separators, and the remaining content of each tube was resuspended in 1.0 mL of PBS.

Prior to analysis, the seven resulting samples were transferred to separate 12×75 mm, polystyrene, snap-cap tubes. The seven samples were then analyzed for fluorescein labeling density by flow cytometry.

The results of the flow cytometry analysis are shown in FIGS. 29-35.

FIGS. 29 and 30 show the unbound particle controls for 0.3 μm-Fl-anti-CD45 scFv and 0.3 μm-Fl-anti-CD45 MoAb in groups 1 and 2, respectively. Particle density was 2.2×10⁷ particles/mL.

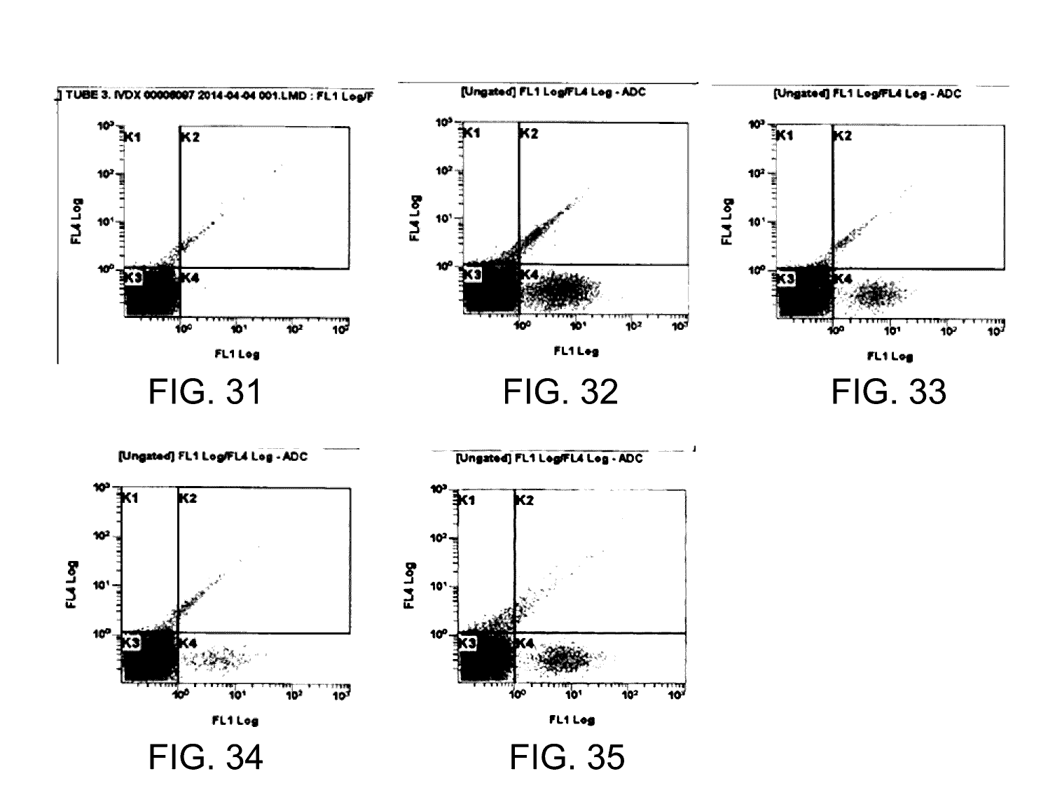

FIG. 31 shows the fluorescent labeling of human leukocytes by the 0.3 μm-Fl-anti-CD45 MoAb probes. Note the increased fluorescent signal compared to the corresponding particle control in group 2 (FIG. 30).

FIGS. 32-34 show the fluorescent labeling of human leukocytes by the 0.3 μm-Fl-anti-CD45 scFv probes from lots A, B, and C, respectively. The labeling response is evidenced by the increase in fluorescent signal compared to the corresponding particle control in group 1 (FIG. 29). Although fluorescent labeling occurred in all 3 groups, it can be seen in FIG. 32 (group 4), that the best fluorescent response was obtained from Lot A of the 0.3 μm-Fl-anti-CD45 scFv conjugate.

In comparison to FIG. 34 (group 6), FIG. 35 (group 7) shows an increased labeling response for Lot C of the 0.3 μm-Fl-anti-CD45 scFv conjugate corresponding to a five-fold increase in the amount of the 0.3 μm-Fl-anti-CD45 scFv conjugate used in group 7.

It is noteworthy that the fluorescent response obtained from the use of each of the 3 Lots of the 0.3 μm-Fl-anti-CD45 scFv conjugate was higher than that obtained from the use of the 0.3 μm-Fl-anti-CD45 MoAb conjugate.

Example 9 demonstrates a shortened procedure time by combining the red blood cell (RBC) lysis step with the antigen labeling step. All 3 Lots of 0.3 μm-Fl-anti-CD45 scFv conjugate successfully labeled the CD45 antigen on human leukocytes.

Example 9 also demonstrates conjugates prepared using the scFv CD45-targeting ligand produced an increased labeling response compared to the conjugate prepared using the monoclonal antibody based CD45-targeting ligand.

Lastly, isolation of labeled cells by magnetic separation was successful. In addition, labeling response is conjugate dose-dependent.

### Example 10

### Rapid Labeling Procedure for the Binding of Fluorescent Magnetic Microspheres to the Folic Acid Receptor on Viable Human Cancer Cells in Whole Blood, and Isolation of Labeled Cells by Magnetic Separation

Folic acid receptor, also known as folate receptor (FR), is a membrane-bound protein with high affinity for binding and transporting folate into cells. Expression of FR is limited in healthy tissues and organs, but it is overexpressed on the vast majority of cancer tissues, including, but not limited to ovary, lung, breast, endometrium, kidney, brain, and others. Therefore, abnormally-high levels of FR expression provide a convenient biomarker for the identification of a wide range of primary and metastatic human cancers. This example describes a novel procedure for the binding of folate-coated fluorescent magnetic microspheres to the folic acid receptor on viable human cancer cells in whole blood, and the isolation and detection of the resulting labeled cells by magnetic separation and flow cytometry. This method provides utility for the development of a rapid assay for the in vitro capture and detection of circulating tumor cells (CTCs) in whole blood and other body fluid samples. The example involved (a) preparation of folic acid receptor targeting probes, (b) incubation of blood sample with folic acid receptor targeting probes, (c) isolation of labeled tumor cells by magnetic separation, and (d) detection and measurement of the labeled tumor cells by flow cytometry.

### (a) Preparation of Folic Acid Receptor-Targeting Magnetic Fluorescent Probes

Paramagnetic particles having a diameter of approximately 0.6 μm, containing a fluorescent yellow dye (480 nm excitation, 520 nm emission), were derivatized with human folate receptor 1 (FOLR1) also known as folate receptor a. Following conjugation, unbound folic acid was removed from the resulting folic acid-coated, magnetic, fluorescent, yellow, 0.6 μm beads by magnetic separation. The resulting purified particles will be referred to as “folic acid receptor-targeting probes.”

(b) Incubation of Blood Sample with Folic Acid Receptor Targeting Magnetic Fluorescent Probes

Human KB cells (ATCC CCL-17), an epithelial cancer cell line which overexpresses FR, were grown in folic acid-deficient tissue culture medium. The KB cells were spiked into a sample of normal human blood, collected from a healthy adult volunteer, to a density of 1×10⁵ cells/mL. A second sample was also prepared, which contained 1×10⁵ KB cells/mL in phosphate buffered saline, pH 7.4 (PBS). A 2 mL volume of each of the two samples was placed in separate 17×100 mm round bottom, snap cap, polypropylene tubes. Folic acid receptor-targeting magnetic fluorescent probes were then added to each of the two samples in the tubes to a density of 1×10⁷, resulting in a ratio of 100 particles per KB cell. To allow the folic acid receptor targeting magnetic fluorescent probes to bind to the FR targets on the surface of the KB cells, the resulting two mixtures were incubated in the dark, at room temperature, for 30 minutes, with gentle inversion (Lab quake rotating mixer).

### (c) Isolation of Labeled Tumor Cells by Magnetic Separation

Each of the 17×100 mm tubes containing the mixtures were placed into magnetic separators (see description of system, above) for 15 minutes to allow the labeled KB cells to be drawn and affixed to the interior sides of the tubes. While leaving the tubes stationary in the magnetic separators, the supernatants were gently aspirated and discarded in order to remove any unbound material. In the case of the blood sample, the unbound material included blood components such as red blood cells, white blood cells, and plasma. To rinse any remaining unbound material from the tubes, a 5 ml volume of wash buffer (phosphate buffered saline, pH=7.4, with 0.1% BSA) was gently added to each tube, and was then aspirated. The tubes were then removed from the magnetic separators and the remaining content of each tube was resuspended in 2 ml of wash buffer. The following table summarizes each of the two prepared samples:

| | No. of 0.6 μm | No. of | Ratio |
|---|---|---|---|
| Test Mixture | beads/ml | cells/ml | (beads:cells) |
| KB cells in PBS + Beads | 1 × 10⁷ | 1 × 10⁵ | 100:1 |
| KB cells in Blood + Beads | 1 × 10⁷ | 1 × 10⁵ | 100:1 |

### (d) Detection and Measurement of the Labeled Tumor Cells by Flow Cytometry

To prepare for analysis, a 0.25 ml volume of each of the resulting two samples was transferred to separate 12×75 mm, polypropylene, snap-cap, assay tubes. To each tube was also added an equal volume (0.25 ml) of Flow-Count Fluorospheres (Beckman Coulter) to allow enumeration of labeled cells. Sample analysis was performed by flow cytometry using the Beckman Coulter Navios System, using a medium flow rate, and a custom-designed instrument protocol configured to enumerate FR-positive cells (FR+).

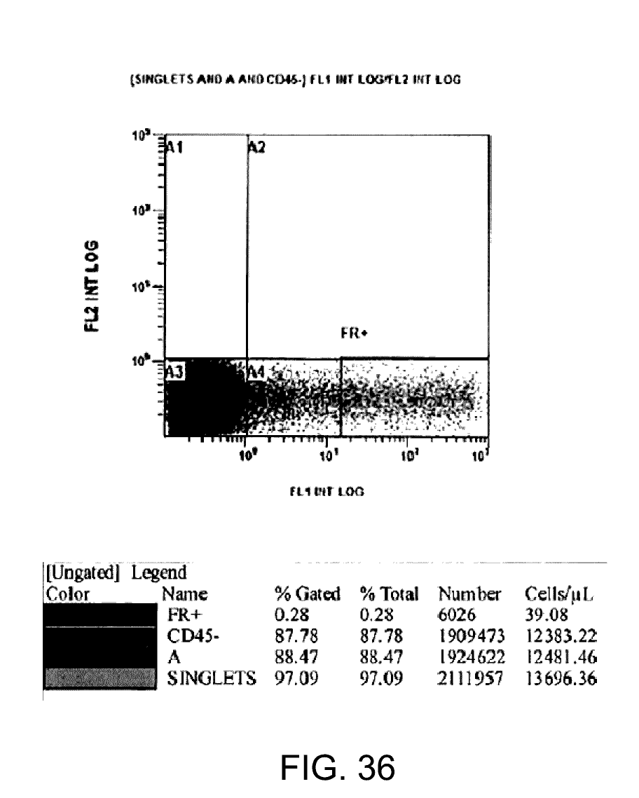

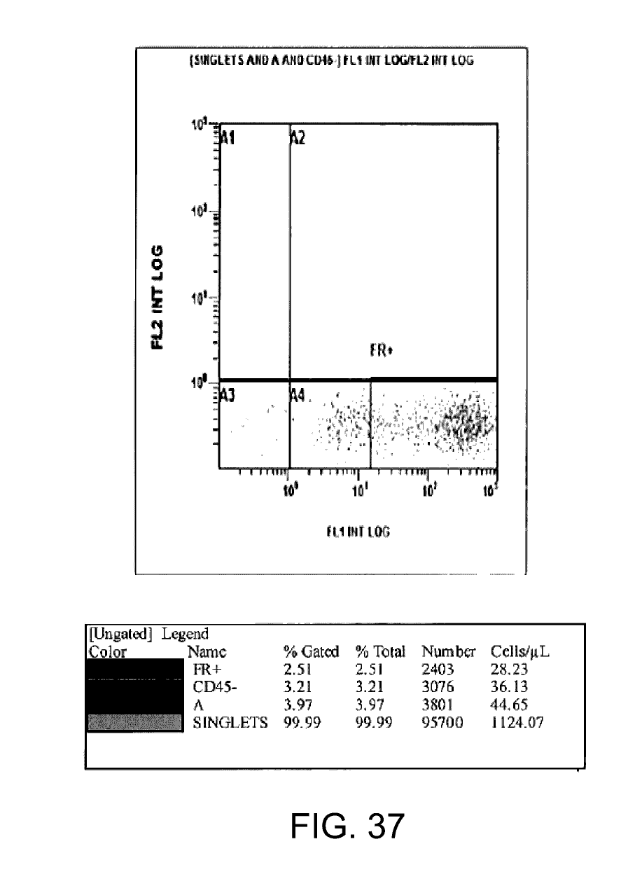

The results of the flow cytometry analysis are shown in FIGS. 36 and 37.

In the sample containing KB cells in Blood+Beads, a density of 39.1 cells/μL were detected for FR+ cells. This corresponds to a density of 3.91×10⁴ cells/mL.

Comparable results were obtained from both samples, thus demonstrating the ability to label, and magnetically separate and recover folate receptor expressing tumor cells from whole blood and other fluids. These results demonstrate the feasibility for the use of the folic acid-coated, magnetic, fluorescent, 0.6 μm beads for the labeling, magnetic separation and detection of folate receptor-expressing tumor cells in a clinical CTC assay format.

### Example 11

### Alternate Embodiment of In Vitro Assay Method

The following is an illustrative example of the magnetic separation procedure for performing an in vitro assay to quantify the number of circulating tumor cells in a blood sample from a cancer patient.

Three (10 mL) Cell Save® preservative tubes are used for the draw, which requires a draw of no less than 20 mL of blood from a patient via venipuncture, using CellSave® preservative tubes following Clinical and Laboratory

Standards Institute (CLSI) procedure GP41-A6 (replaces document H03-A6) for circulating tumor cell (CTC) analysis within 48 hrs. CellSave® tubes are optimized to stabilize circulating tumor cells (CTCs) for up to 96 hours at room temperature, which allows shipment of samples from remote locations via postal service or other courier for analysis. The tubes are filled until blood flow stops. If 20 mL is not collected from one IV stick, it is permissible to use another IV draw location. Immediately mix by gently inverting the tubes 8 times to prevent, or at least minimize the likelihood of clotting. Samples are transported and stored at temperatures of 15-30° C. (59-86° F.), as refrigeration of the samples prior to processing could adversely affect sample integrity. To mitigate the risk of refrigeration, blood can be shipped in containers layered with absorbent material and labeled with a warning to keep the container at room temperature (15-30° C.), for example.

Upon arrival at the diagnostic laboratory, refrigerated RBC lysis buffer, Flow-Count beads (BD), fluorescent conjugate of folic acid (“IVD001”) reagent, anti-CD45 magnetic fluorophore microparticles and phosphate buffered saline (PBS) are warmed to room temperature, for at least 20 minutes, as appropriate. Flow-Count beads (BD), fluorescent conjugate of folic acid (IVD001) reagent, anti-CD45 magnetic fluorophore microparticles reagents should be shielded from light through proper storage or covering with a reflective material such as a metallic foil, for example.

The received CellSave® tubes containing the donated blood are gently vortexed for at least 5 seconds to evenly suspend the blood cells. The blood is pooled into a 50 mL sterile conical tube and gently vortex for 10 seconds, before 10 mL of blood is transferred to either a 50 mL sterile yellow cap (TPP—Techno Plastic Products) polypropylene conical tube or to a Falcon blue cap (BD Biosciences) polypropylene conical tube using a 10 mL serological pipette and pipette aid and by pipetting from the bottom center of the blood specimen. This step will be done for each of two 50 mL conical tubes.

For each assay performed, positive and negative control samples are also to be prepared using the provided control suspensions, which can be comprised of either fixed cell controls or antigen-coated polystyrene microspheres, for example. The two controls are to be processed in a manner identical to the clinical blood samples. 20 mL of RBC lysis buffer is added to each 50 mL conical tube using a 25 mL sized serological pipette and pipette aid, vortex the tube for 10 seconds.

### First Embodiment

After vortexing a container of magnetic fluorophore microparticles conjugated anti-CD45 scFv for 10 seconds, 50 μL of the working suspension (contains 1×10¹¹ particles/mL) is added to each of the two 50 mL conical tubes by pipetting, and the tubes are then gently vortexed for 10 seconds. The blood specimens are to be incubated at room temperature for 5 min. Periodic agitation of the tube during incubation will improve the lysis effect. After incubation, the blood specimens are transferred from each of the two 50 mL conical tubes, and 10 mL of each is transferred to six blue cap, 15 mL sterile tubes.

### Second Embodiment

Instead of incubating according to the preceding First Embodiment, a rare-earth rod magnet can optionally be inserted into each of the 50 mL conical tubes. Incubating the rods from 1 to 5 minutes will capture a substantial portion of the magnetic fluorophore microparticles conjugated anti-CD45 scFv with the captured leukocytes. Once the magnetic rods are removed, they may be cleaned and ready to use again. Sintered Neodymium-Iron-Boron (NdFeB) rods are plated with Ni—Cu—Ni (Nickel) for corrosion resistance but can also include an epoxy or other plastic coatings. These rod magnets are magnetized through their length and possess an individual pull force of approximately 4 lbs. Other magnetic rods with various dimensions and pull force properties are also available. Once the rods have been removed, the caps are replaced on the conical tubes.

After incubation according to the First Embodiment or the Second Embodiment, transfer the resulting blood specimens from each of the two 50 mL conical tubes, adding 10 mL to each of six blue cap, 15 mL sterile tubes. Insert each blue cap tube into the magnetic separator 10 for five (5 min.) minutes. Without removing the blue cap tubes from the magnetic separator 10, collect all fluid from each blue cap tube using a 5 mL serological pipette and pipette aid and add to a sterile 50 mL conical tube. Place contents of three 15 mL sterile tubes into a single 50 mL conical tube.

Tighten the cap of the each conical tube and centrifuge the tubes at 1,000×G for 5 min using a swing-bucket centrifuge. The centrifuge should impart a relative centrifugal force of at least 1,000 RCFs. Decant the supernatant as the cancer cells will reside in the pellet of each 50 mL conical tube.

Suspend each pellet by adding 0.5 mL of wash/dilution buffer (phosphate buffered saline containing bovine serum albumin as a blocking agent) to each 50 mL conical tube and vortex gently for 5 seconds. Pool each of the resuspended pellets for a total volume of 1.0 mL.

Vortex the vial of IVD001 for 5 seconds, add 5 μL of the IVD001 working solution (10 μM) per 1 mL sample volume and 250 μL of Beckman Coulter Flow-Count™ fluorospheres, vortex gently for 5 seconds and incubate for 5 minutes while shielding from light.

Return remaining IVD001, magnetic fluorophore microparticles conjugated to anti-CD45 scFv, Beckman Coulter Flow-Count™ fluorospheres, and refrigerate them at 2°-8° C. immediately. The addition of IVD001 conjugated to magnetic microparticles is believed to facilitate the collection of cancer cells and cancer stem cells, expansion of the cell population and characterization of the cancer cell and its corresponding susceptibility to a specific chemotherapy.

The flow cytometry analysis tubes are prepared and labeled with patient ID numbers. Identify by sample number/patient ID, positive control and negative control on all tubes. Flow cytometer tubes are typically 12 mm×75 mm with round bottoms.

The entire 1 mL volume of each processed patient sample and control is transferred into separate microdilution tubes, which are then slid into the appropriately-labeled flow cytometry analysis tube. The samples are transferred to the microdilution tubes in order raise the fluid level of each sample to ensure there will be sufficient volume for the flow cytometer analysis.

Samples are to be loaded onto the carousel for sequential analysis by flow cytometry. Vortexing is needed before flow cytometry analysis, if cell settling is observed in the tubes. Samples are analyzed at medium flow rate, 10M (million) max events, and an acquisition time of 15 minutes. Total assay time for one patient's specimen is approximately 150 min, or less. Flow cytometer processing per flow cytometry analysis tube can be approximately 15 min, or less. The total time required to process the diagnostic assay and flow cytometer analysis can be approximately 180 min, or less.

IVD001 is a fluorescent conjugate of folic acid, which binds to the folate receptor of cells. More specifically, IVD001 comprises pteroyl-γ-glutamic acid-cysteine Oregon Green®488. IVD-001 is supplied as a solution (10 μM) in individual 1.0 mL sample vials. This concentration will be sufficient to perform approximately 200 assays.

Anti-CD45 scFv: anti-CD45 scFv conjugated to a fluorophore embedded magnetic microparticle. A working suspension can include 1×10¹¹ particles/mL suspended in phosphate buffered saline, pH 7.4.

Flow Count™ Fluorospheres (Beckman Coulter)—*20 ml size. Flow-Count Fluorospheres are a suspension of fluorospheres used to determine absolute counts on the flow cytometer. Each fluorosphere contains a dye that has a fluorescent emission range of 525 nm to 700 nm when excited at 488 nm. They have uniform size and fluorescence intensity, and an assayed concentration, allowing a direct determination of absolute counts.

Assay Controls. Supplied as either fixed cells or antigen-coated polystyrene microspheres. Positive control will consist of particles (cells or microspheres) having a high density of surface-exposed folate receptors, and no CD45. Negative control will consist of particles having surface-exposed CD45, and no folate receptors.

Illustrative embodiments have been described, hereinabove. It will be apparent to those skilled in the art that the above devices and methods may incorporate changes and modifications without departing from the general scope of this invention. It is intended to include all such modifications and alterations within the scope of the present invention. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An assay method comprising:
contacting human blood cells in a liquid medium with a conjugate comprising a fluorescent magnetic particle derivatized with a molecule that demonstrates specificity for a target on a surface of malignant blood cells included in the liquid medium to form labeled malignant blood cells;
exposing the liquid medium including the labeled malignant blood cells to a magnetic field to separate the labeled malignant blood cells from unlabeled blood cells in the liquid medium;
in the presence of the magnet field, removing at least a portion of the liquid medium to isolate the labeled malignant blood cells separated by the magnetic field; and
introducing a sample comprising at least a portion of the labeled malignant blood cells separated by the magnetic field into a flow cytometer to quantify the labeled malignant blood cells present in the sample.

2. The assay method of claim 1 further comprising lysing red blood cells present in the liquid medium.

3. The assay method of claim 2, wherein said lysing occurs prior to said introduction of the sample into the flow cytometer.

4. The assay method of claim 2, wherein said lysing is performed concurrently with said contacting the human blood cells with the conjugate.

5. The assay method of claim 1 further comprising washing the labeled malignant blood cells prior to said introducing the sample into the flow cytometer.

6. The assay method of claim 1 further comprising re-suspending the labeled malignant blood cells in a buffer solution to form the sample introduced to the flow cytometer.

7. The assay method of claim 1, wherein said liquid medium including the labeled malignant blood cells is exposed to a magnetic field for at least five (5 min.) minutes.

8. The assay method of claim 1, wherein the molecule that demonstrates specificity for the target comprises anti CD45 scFv.

9. The assay method of claim 1, wherein the molecule that demonstrates specificity for the target comprises at least one of: polyclonal, monoclonal, scFv, aptamer, lectin, peptides.

10. The assay method of claim 1, wherein the fluorescent magnetic particle is formed from a material selected from the group consisting of: polystyrene, Poly(methyl methacrylate (PMMA), Poly(lactic-co-glycolic) acid (PLGA), and Polycaprolactone (PCL).
